(19) United States
(12) Patent Application Publication
(10) Pub. No.: US 2007/0099888 A1
(43) Pub. Date: May 3, 2007

(54) Inflammation Inhibitor Comprising Zinc Salt of Acylamino Acid

(76) Inventors: Keiji Iwasaki, Kawasaki-shi (JP); Manabu Kitazawa, Kawasaki-shi (JP); Yoshinobu Takino, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/610,736

(22) Filed: Dec. 14, 2006

(30) Foreign Application Priority Data
Jun. 18, 2004 (JP) 2004-180880
Jun. 15, 2005 (WO) PCT/JP05/11404

(51) Int. Cl. A61K 31/555; A61K 31/315

(52) U.S. Cl. 514/185; 514/494

(57) ABSTRACT

An inflammation inhibitor for the skin is provided containing a zinc salt of an acylamino acid and further, a cosmetic containing the inflammation inhibitor.

This application claims priority under 35 U.S.C. §120 to PCT/JP2005/011404, filed Jun. 15, 2005, and under 35 U.S.C. § 19(a) to Japanese patent application 2004-180880, filed Jun. 18, 2004, the entirties of both of which are hereby incorporated by reference.

### BACKGROUND OF THE INVENTION

1. Field of the Invention

The present invention relates to an inflammation inhibitor, which is useful for preventing, delaying, improving, or treating an inflammatory disease, skin damage, or a disease caused by inflammation. The present invention further relates to a composition for external use containing the inflammation inhibitor as an active ingredient, such as a cosmetic or a skin preparation.

2. Brief Description of the Related Art

Infectious substances such as bacteria and viruses, antigenic substances which cause allergic reactions, stimulating factors which cause tissue damage, ultraviolet light which can induce skin cancer or accelerated aging of the skin, and the like, are all factors which cause inflammation of the skin. Due to these factors, the skin can be damaged, and therefore, inhibiting excessive inflammatory reactions may lead to alleviation of skin damage.

In recent years, studies on the mechanism of action of skin inflammation have progressed, and it has been reported that inflammatory cytokines, such as IL-1α, TNF-α, or an extracellular matrix metalloproteinase such as collagenase, play a role in inflammation. The expression of these cytokines or extracellular matrix metalloproteinase is regulated at the gene level by a transcription regulator, such as NF-κB or AP-1. For example, it has been reported that when the skin is exposed to ultraviolet light included in sunlight, NF-κB or AP-1 are activated in the skin cells, which accelerates skin aging (for example, Nature, Vol. 379, pp. 335-339, 1996). Accordingly, if these inflammatory factors can be inhibited, skin disorders caused by the various factors may be alleviated.

Various substances have been reported which inhibit the inflammatory factors which cause skin damage. For example, an antioxidant substance, such as N-acetyl-L-cysteine, inhibits the activation of NF-κB or AP-1 in epidermal cells (for example, Free Rad. Biol. Med., Vol. 26, pp. 174-183 and FEBS Letters, Vol. 384, pp. 92-96, 1996). However, the effective concentration is 10 mM to 30 mM, but still has insufficient effects, or the toxicity to a cell is strong, and the like. Other than N-acetyl-L-cysteine, it has also been reported that retinoic acid inhibits the activation of AP-1 and the expression of an extracellular matrix metalloproteinase (for example, Nature, Vol. 379, pp. 335-339, 1996). However, retinoic acid has side effects such as stimulation and peeling of the skin, so its use is limited. On the other hand, skin damage which is caused by ultraviolet light can be inhibited by induction of metallothionein, which is an endogenous antioxidant substance present in the skin, due to a zinc salt of an amino acid or a zinc salt of a fatty acid (for example, International publication WO 00/44341 and International publication WO 93/14748). However, the effects of these compounds are insufficient, and also it is not known whether activation of a gene transcription factor such as AP-1, which is a major factor of inflammation caused by ultraviolet light, is inhibited by these compounds. Furthermore, zinc oxide, which is related to zinc, prevents diaper rash (for example, Eur. Acad. Dermatol. Veneol. 15 (Suppl. 1) pp. 5-11, 2001); however, its effect is also insufficient, and also it is not known whether activation of a gene transcription factor such as AP-1, which is a major factor of inflammation by ultraviolet light, is inhibited by this substance. There is a possibility that zinc acetate may prevent arteriosclerosis by inhibiting the activity of AP-1, which increases when vascular endothelial cells are cultured in the absence of zinc (J. Am. Clloge Nutrt. 16(5), 411-417, 1997); however, it is not known whether or not it inhibits inflammation of the skin.

### SUMMARY OF THE INVENTION

In view of the above background art, an object of the present invention is to provide a cosmetic or a skin preparation for external use containing as an active ingredient an inflammation inhibitory component which prevents, improves, or treats inflammatory disorder(s) of the skin by inhibiting activation of a gene transcription factor of an extracellular matrix metalloproteinase.

The present inventors have made intensive studies in order to achieve the above object, and as a result, they have found that a zinc salt of an acylamino acid represented by the following general formula (I) inhibits the activation of a gene transcription factor of an extracellular matrix metalloproteinase, and thus the present invention has been completed. It is an object of the present invention to provide an inflammation inhibitor comprising zinc salts of an acylamino acid comprising: 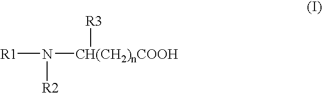
wherein, in the formula (I), R1 represents an acyl group having 2 to 22 carbon atoms, R2 represents a hydrogen atom or a linear or branched alkyl group having 1 to 6 carbon atoms, R3 represents a side chain selected from the group consisting of valine, leucine, isoleucine, phenylalanine, methionine, tryptophan, asparagine, glutamine, serine, tyrosine, aspartic acid, glutamic acid, lysine, arginine, histidine, and hydrogen, and n represents an integer of 0 or 1.

It is a further object of the present invention to provide that when R2 is a hydrogen atom, n is preferably 0, and the inflammation inhibitor comprises a zinc salt of an acylamino acid comprising: 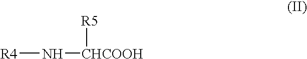
wherein, in the formula (II), R4 represents an acyl group having 2 to 22 carbon atoms, R5 represents a side chain selected from the group consisting of valine, leucine, isoleucine, phenylalanine, methionine, tryptophan, asparagine, glutamine, serine, tyrosine, aspartic acid, glutamic acid, lysine, arginine, histidine, and hydrogen.

It is a further object of the present invention to provide a preventive or therapeutic agent for an skin inflammatory disease comprising the inflammation inhibitor as described above.

It is a further object of the present invention to provide a cosmetic additive or a skin preparation for external use comprising the inflammation inhibitor described above.

It is an even further object of the invention to provide a method of preventing, delaying, improving or treating a skin disease due to aging or ultraviolet light comprising applying to the skin a composition comprising an inflammation inhibitor as described above.

According to the present invention, an inflammation inhibitor that can be used for a cosmetic or the like can be obtained.

### DESCRIPTION OF THE PREFERRED EMBODIMENTS

Hereinafter, the present invention will be described in detail.

First, specific examples of the compound according to the present invention will be described. In an acylamino acid represented by the following general formula (I): 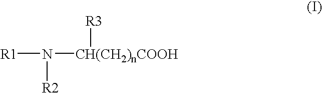
examples of R1 include an acetyl group, a propioyl group, an isopropiol group, an n-butyloyl group, an isobutyloyl group, a sec-butyloyl group, a tert-butyloyl group, an n-amyloyl group, a sec-amyloyl group, a tert-amyloyl group, an isoamyloyl group, an n-hexyloyl group, a cyclohexyloyl group, an n-heptanoyl group, an n-octanoyl group, a 2-ethylhexyloyl group, a nonyoyl group, an isononyoyl group, a decanoyl group, an isodecanoyl group, an undecanoyl group, a lauroyl group, a tridecanoyl group, an isotridecanoyl group, a myristoyl group, a palmitoyl group, an isopalmitoyl group, a stearoyl group, an isostearoyl group, an oleoyl group, a docosanoyl group, a cocoyl group, and the like. Among these, an n-octanoyl group, a decanoyl group, a lauroyl group, a myristoyl group, a palmitoyl group, a stearoyl group, an oleoyl group, a cocoyl group, and the like, are preferred, and furthermore, a lauroyl group, a myristoyl group, a palmitoyl group, and a cocoyl group are particularly preferred.

Examples of R2 include a methyl group, an ethyl group, a propyl group, an isopropyl group, an n-butyl group, an isobutyl group, a sec-butyl group, a tert-butyl group, an n-amyl group, a sec-amyl group, a tert-amyl group, an isoamyl group, an n-hexyl group, a cyclohexyl group, a hydrogen atom and the like. Among these, a methyl group, an ethyl group, a propyl group, an isopropyl group, and a hydrogen atom are preferred, and particularly preferred are a methyl group and a hydrogen atom.

Examples of R3 include a side chain of valine, leucine, isoleucine, phenylalanine, methionine, tryptophan, asparagine, glutamine, serine, tyrosine, aspartic acid, glutamic acid, lysine, arginine, histidineor a hydrogen atom, and the like. Among these, a side chain of valine, leucine, isoleucine, serine, aspartic acid, glutamic acid, histidine, or a hydrogen atom are preferred, and furthermore, a side chain of glutamic acid, histidine or a hydrogen atom are particularly preferred. The tertiary structure of the amino acid moiety may be the L-form, D-form and/or the DL-form; however, it is preferably the L-form. n represents an integer of 0 or 1.

A preferred combination of substituents is that when R1 is an n-octanoyl group, a decanoyl group, a lauroyl group, a myristoyl group, a palmitoyl group, a stearoyl group, an oleoyl group, or a cocoyl group, R2 is a methyl group, an ethyl group, a propyl group, an isopropyl group, or a hydrogen atom, and R3 is a side chain of valine, leucine, isoleucine, serine, aspartic acid, glutamic acid, histidine, or a hydrogen atom, and particularly preferably, R1 is a lauroyl group, R2 is a hydrogen atom, and R3 is a side chain of glutamic acid, histidine, or a hydrogen atom, etc. Furthermore, when R2 is a hydrogen atom, n is preferably 0, and the compound is represented by the following general formula (II).

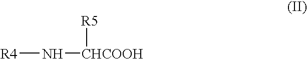

In this embodiment, the definition of R4 is the same as that of the above R1, and the definition of R5 is the same as that of the above R3. However, the present invention is not limited to these.

The inflammation inhibitor of the present invention can be orally or parenterally administered; however, it is preferably administered by applying it to the surface of the skin. When either a zinc salt of an acylamino acid represented by the above general formula (I) or a zinc salt of the above organic acid is blended in a cosmetic or a skin preparation as an active ingredient for preventing or improving an inflammatory skin disease or inflammatory skin damage, the blending amount thereof is 0.01% to 10% by weight, preferably 0.1% to 5% by weight. When the blending amount is less than 0.01% by weight, inhibition of inflammation does not sufficiently occur. When it exceeds 10%, a scraping feeling against the skin occurs, and therefore, too little or too much of the zinc salt as defined above is not preferred. When a zinc salt of an acylamino acid represented by the above general formula (I) is blended in a cosmetic or a skin preparation, components or excipients which are generally used in cosmetics or skin preparations for external use can be added in an amount which does not inhibit the effect of the present invention.

Examples of components which are generally used in a cosmetic or a skin preparation for external use include an antioxidant, an anti-inflammatory agent, an ultraviolet absorber, a whitening agent, a cell activator, a moisturizing agent, a metal chelating agent, an oleaginous material, a surfactant, a solvent, a polymeric substance, a powdery substance, a dye, a fragrance, a transdermal absorption promoting agent, a steroid hormone, and the like.

Examples of the antioxidant include the vitamin A group of compounds, including retinol, dehydroretinol, retinol acetate, retinol palmitate, retinal, retinoic acid, vitamin A oil, and derivatives thereof, carotenoids such as α-carotene, 13-carotene, γ-carotene, cryptoxanthin, astaxanthin, fucoxanthin, and derivatives thereof, the vitamin B group of compounds, including pyridoxine, pyridoxal, pyridoxal-5-phosphate ester, pyridoxamine, and derivatives thereof, the vitamin C group including ascorbic acid, sodium ascorbate, ascorbyl stearate, ascorbyl palmitate, ascorbyl dipalmitate, ascorbate magnesium phosphate, and derivatives thereof, the vitamin D group including ergocalciferol, cholecalciferol, 1,25-dihydroxy-cholecalciferol, and derivatives thereof, the vitamin E group including α-tocopherol, β-tocopherol, γ-tocopherol, δ-tocopherol, α-tocotrienol, β-tocotrienol, γ-tocotrienol, δ-tocotrienol, tocopherol acetate, tocopherol nicotinate, and derivatives thereof, trolox and derivatives thereof, dihydroxytoluene, butylhydroxytoluene, butylhydroxyanisole, dibutylhydroxytoluene, α-lipoic acid, dehydrolipoic acid, glutathione, and derivatives thereof, uric acid, erythorbic acids such as erythorbic acid and sodium erythorbate, and derivatives thereof, gallic acid and gallic acid derivatives such as propyl gallate, rutin and rutin derivatives such as α-glycosyl-rutin, tryptophan and derivatives thereof, histidine and derivatives thereof, cysteine derivatives such as N-acetylcysteine, N-acetylhomocysteine, N-octanoylcysteine, and N-acetylcysteine methyl ester, cystine derivatives described in WO/0021925 such as N,N′-diacetylcystine dimethyl ester, N,N′-dioctanoylcystine dimethyl ester, and N,N′-dioctanoylhomocystine dimethyl ester, carnosine and derivatives thereof, homocamosine and derivatives thereof, anserine and derivatives thereof, carcinine and derivatives thereof, dipeptide or tripeptide derivatives including histidine and/or tryptophan and/or histamine, flavonoids such as flavanone, flavone, anthocyanin, anthocyanidine, flavonol, quercetin, quercitrin, myricetin, fisetin, hamamelitannin, catechin, epicatechin, gallocatechin, epigallocatechin, epicatechin gallate, and epigallocatechin gallate, tannic acid, caffeic acid, ferulic acid, protocatechuic acid, chalcone, oryzanol, carnosol, sesamol, sesamin, sesamolin, zingerone, curcumin, tetrahydrocurcumin, clovamide, deoxyclovamide, shogaol, capsaicin, vanillyl amide, ellagic acid, bromophenol, flavogracin, melanoidin, riboflavin, riboflavin butyrate ester, flavin mononucleotide, flavin adenine nucleotide, ubiquinone, ubiquinol, mannitol, bilirubin, cholesterol, ebselen, selenomethionine, ceruloplasmin, transferrin, lactoferrin, albumin, bilirubin, superoxide dismutase, catalase, glutathione peroxidase, metallothionein, o-phosphono-pyridoxylidene rhodamine, and N-(2-hydroxybenzyl)amino acid described in U.S. Pat. No. 5,594,012 and derivatives thereof, and N-(4-pyridoxylmethylene)amino acid and derivatives thereof, and the like.

Examples of the anti-inflammatory agent include phenylbutazone, indomethacin, ibuprofen, ketoprofen, allantoin, guaiazulene, resorcin, hydrocortisone, prednisolone, methylprednisolone, dexamethasone, triamcinolone, triamcinolone acetonide, fludoxycortide, clobetasone, clobetasol and esters of these steroids, ketal, acetal and hemiacetal derivatives, flufenamic acid, bufexamac, naploxen, fluviprofen, fenbufen, tenoxicam, piroxicam, mefenamic acid, salicylic acid, salicylate derivatives such as sodium salicylate, methyl salicylate, and glycol salicylate, D-panthenol and derivatives thereof, glycyrrhizic acid and derivatives thereof such as methyl glycyrrhizinate, and dipotassium glycyrrhizinate, glycyrrhetinic acid and derivatives thereof such as glyceryl glycyrrhate, stearyl glycyrrhate and glycyrrhetinyl stearate, chondroitin sulfate, ε-aminocaproic acid, sodium diclofenac, tranexamic acid, diphenhydramine hydrochloride, chlorpheniramine maleate, ichthammol, γ-oryzanol, thianthol, sodium copper chlorophyllin, Angelica keiskei extract, Arnica Montana extract, aloe extract, Bistorta major extract, turmeric extract, Hypericum erectum extract, German chamomile extract, Glycyrrhiza glabra extract, Lonicera japonica extract, Nasturtium officinale extract, Symphytum officinale extract, Acanthopanax gracilistylus extract, salvia extract, Lithospermum root extract, Perilla extract, white birch extract, tea extract, Angelica acutiloba extract, Calendula officinalis extract, elderberry extract, Typha angustifolia extract, Sapindus mukurossi extract, Artemisia extract, eucalyptus extract, Astragalus sinicus extract, zinc oxide, and the like.

Examples of the ultraviolet absorber include cinnamic acid-based ultraviolet absorbers such as p-methoxycinnamate-2-ethylhexyl, isopropyl p-methoxycinnamate, sodium p-methoxycinnamate, potassium p-methoxycinnamate, p-methoxycinnamate-2-ethoxyethyl, p-methoxyhydrocinnamate diethanolamine salt, di-p-methoxycinnamate-mono-2-ethylhexanoate glyceryl, octyl methoxycinnamate and methyl diisopropylcinnamate, benzophenone-based ultraviolet absorbers such as 2-hydroxy-4-methoxybenzophenone, 2-hydroxy-4-methoxybenzophenone-5-sulfuric acid, 2-hydroxy-4-methoxybenzophenone-5-sulfate sodium, dihydroxybenzophenone, 2,4-dihydroxybenzophenone, 2,2′-dihydroxy-4,4′-dimethoxybenzophenone, 2,2′-dihydroxy-4-methoxybenzophenone, dihydroxydimethoxybenzophenonesulfate sodium, 2,2′,4,4′-tetrahydroxybenzophenone, and 2-hydroxy-4-n-octoxybenzophenone, benzoic acid-based ultraviolet absorbers such as p-aminobenzoic acid, sodium p-aminobenzoate, ethyl p-aminobenzoate, butyl p-aminobenzoate, p-dimethylaminobenzoate-2-ethylhexyl, amyl p-dimethylaminobenzoate, glyceryl p-aminobenzoate, and amyl p-aminobenzoate, salicylic acid-based ultraviolet absorbers such as salicylate-2-ethylhexyl, salicylate triethanolamine, homomenthyl salicylate, salicylate dipropylene glycol, methyl salicylate, salicylate ethylene glycol, phenyl salicylate, amyl salicylate, benzyl salicylate, isopropylbenzyl salicylate, myristyl salicylate, and potassium salicylate, dibenzoylmethane-based ultraviolet absorbers such as 4-tert-butyl-4′-methoxydibenzoylmethane, 4-isopropyldibenzoylmethane, 4-methoxydibenzoylmethane, and 4-tert-butyl-4′-hydroxydibenzoylmethane, urocanic acid-based ultraviolet absorbers such as urocanic acid, and ethyl urocanate, menthyl-O-aminobenzoate, 2-phenyl-benzimidazole-5-sulfuric acid, 2-phenyl-5-methylbenzoxazole, 3-(4-methylbenzylidene)camphor, 2-ethylhexyl-2-cyano-3,3′-diphenyl acrylate, 2-ethyl-2-cyano-3,3′-diphenyl acrylate, 2-(2′-hydroxy-5-methylphenyl)benzotriazole, methyl anthranilate, ethyl anthranilate, menthyl anthranilate, 2,4,6-tris[4-(2-ethylhexyloxycarbonyl)anilino]-1,3,5-triazine, 3,3′-(1,4-phenylenedimethylidene)bis(7,7-dimethyl-2-oxo-bicyclo[2.2.1]heptane-1-methanes ulfonic acid) (Mexoryl SX), titanium oxide, zirconium oxide, cerium oxide, zinc oxide, and the like.

Examples of the whitening agent include tyrosinase inhibitors, endothelin antagonists, α-MSH inhibitors, glabridin, glabrene, liquiritin, isoliquiritin, ellagic acid and derivatives thereof, kojic acid and derivatives thereof, hydroquinone such as arbutin and derivatives thereof, cysteine and derivatives thereof, the vitamin C group including ascorbic acid, sodium ascorbate, ascorbyl stearate, ascorbyl palmitate, ascorbyl dipalmitate, and ascorbate magnesium phosphate and derivatives thereof, glutathione and derivatives thereof, resorcin and derivatives thereof, neoagarobiose, agarose oligosaccharide, asparagus extract, Althaea extract, Bistorta extract, Artemisiae Capillaris extract, Pisum bean extract, rose fruit extract, Scutellaria root extract, Ononis spinosa extract, seaweed extract, Pyracantha fortuneana extract, Glycyrrhiza glabra extract, raspberry extract, Sophora flavescens extract, unrefined sugar extract, Spatholobus suberectus Dunn extract, Acanthopanax gracilistylus extract, wheat germ extract, Asiasari Radix extract, crataegus extract, Cassia mimosoides extract, peony root extract, white lily extract, Inula britannica extract, Mori cortex extract, soybean extract, placenta extract, Aralia elata extract, tea extract, Angelica acutiloba extract, molasses extract, Rosa multiflora Thunb extract, Ampelopsis japonica Makino extract, grape seed extract, Fagus crenata extract, Flodemannita extract, hops extract, Rosa rugosa extract, Chaenomeles sinensis extract, Saxifraga stolonifera extract, Coix seed extract, Momordica grosvenori extract, and the like.

Examples of the cell activator include nucleic acid-related substances such as deoxyribonucleic acids, adenylic acid, ribonucleic acids, cyclic AMP, cyclic GMP, flavin adenine nucleotide, guanine, adenine, cytosine, thymine, xanthine, caffeine, and theophylline and derivatives thereof, the vitamin A group including retinol, dehydroretinol, retinol acetate, retinol palmitate, retinal, retinoic acid and vitamin A oil and derivatives thereof, carotenoids such as α-carotene, β-carotene, γ-carotene, cryptoxanthin, astaxanthin and fucoxanthin, and derivatives thereof, the vitamin B group including pyridoxine, pyridoxal, pyridoxal-5-phosphate ester and pyridoxamine and derivatives thereof, the vitamin C group including ascorbic acid, sodium ascorbate, ascorbyl stearate, ascorbyl palmitate, ascorbyl dipalmitate and ascorbate magnesium phosphate and derivatives thereof, the vitamin D group including ergocalciferol, cholecalciferol and 1,25-dihydroxy-cholecalciferol and derivatives thereof, the vitamin E group including α-tocopherol, β-tocopherol, γ-tocopherol, δ-tocopherol, α-tocotrienol, β-tocotrienol, γ-tocotrienol, δ-tocotrienol, acetate tocopherol and nicotinate tocopherol and derivatives thereof, trolox and derivatives thereof, hinokitiol, cepharanthine, α-linolenic acid, γ-linolenic acid, eicosapentaenoic acid and derivatives thereof, organic acids selected from glycolic acid, succinic acid, lactic acid and salicylic acid and derivatives thereof, estradiol and derivatives thereof, silk protein and decomposition products thereof, or derivatives thereof, hemoglobin or decomposition products thereof, lactoferrin or decomposition products thereof, animal-derived extracts such as royal jelly, placenta extract, calf blood extract solution, serum protein-free extract, spleen extract, egg ingredients, cock's crest extract, shell extract, shell fish meat extract, Mollusca extract and fish meat extract, microorganism-derived extracts such as fermentation products and metabolic products, plant extracts such as asparagus extract, apricot extract, ginkgo extract, phellodendron extract, barley extract, Panax ginseng extract, orange extract, kiwi extract, cucumber extract, shiitake extract, Equisetum arvense extract, Swertia japonica extract, Zizyphi fructus extract, Capsicum annuum extract, Calendula officinalis extract, carrot extract, garlic extract, Parthenium hysterophorus extract, grape seed extract, beech bud extract, peach extract, eucalyptus extract, Ganoderma lucidum extract, lettuce extract, lemon extract, rosemary extract, and the like.

Examples of the moisturizing agent include mucopolysaccharides, proteins or decomposition products thereof and derivatives thereof, soybean or egg-derived phospholipid, glycolipid, ceramide, mucin, honey, sugars such as erythritol, maltose, maltitol, xylitol, xylose, pentaerythritol, fructose and dextrin and derivatives thereof, acidic polysaccharides such as hyaluronic acid, urea, amino acids such as asparagine, aspartic acid, alanine, arginine, isoleucine, ornithine, glutamine, glycine, glutamic acid, cysteine, cystine, citrulline, threonine, serine, tyrosine, tryptophan, theanine, valine, histidine, hydroxylysine, hydroxyproline, pyrrolidonecarboxylic acid, proline, phenylalanine, methionine and lysine and derivatives thereof, D-panthenol, whey protein, Angelica keiskei extract, avocado extract, almond extract, Althaea extract, Arnica montana extract, aloe extract, strawberry extract, Ceratonia siliqua extract, rice extract, Artemisia capillaris Thunb extract, fennel extract, turmeric extract, Malva sylvestris extract, Perilla frutescens extract, Scutellaria root extract, Coptis rhizome extract, Lamium album var. barbatum extract, Hypericum erectum extract, Ononis spinosa extract, olive oil, seaweed extract, cacao butter, German chamomile extract, Avena fatua extract, Garcinia Cambodia extract, Glycyrrhiza glabra extract, raspberry extract, Hedera rhombea extract, Lonicera japonica extract, gardenia extract, Sasa veitchii extract, grape fruit extract, Sophora root extract, Nasturtium officinale extract, Gentiana lutea extract, Geranium thunbergii extract, Arctium lappa extract, Clematis apiifolia extract, sesame extract, wheat extract, Symphytum officinale extract, Asiasarum root extract, Cactales extract, Saponaria officinalis extract, Salvia extract, Crataegus extract, Butyrospermum parkii extract, Perilla extract, Rehmannia glutinosa extract, Spiraea extract, peony root extract, ginger extract, white birch extract, Malva sylvestris extract, Cnidium rhizome extract, Mori cortex extract, soybean extract, Thymus vulgaris extract, tea extract, camellia extract, Angelica radix extract, corn extract, plant worm extract, Houttuynia cordata Thunb extract, tormentilla extract, Lupinus extract, Ophiopogon tuber extract, parsley extract, Mentha extract, Mentha spicata extract, Mentha piperita extract, Hamamelis extract, rose extract, hinoki extract, sunflower extract, grape extract, Butchers bloom extract, prune extract, Luffa aegyptiaca extract, Tilia extract, Paeonia extract, hops extract, jojova oil, borage oil, macadamia nut extract, pine extract, Cyclonia oblonga extract, Aesculus hippocastanum extract, Sapindus mukurossi extract, Lithospermum erythrorhizon extract, meadowhome oil, melissa extract, Rodgersia podophylla extract, Saxifraga stolonifera extract, Citrus junos extract, lily extract, Coix seed extract, lime extract, Momordica grosvenori extract, lavender extract, apple extract, gentian extract, Astragalus sinicus extract, Sanguisorba extract, alkali simple thermal spring, deep water, and the like.

Examples of the metal chelating agent include malic acid, citric acid, salicylic acid, tartaric acid, gluconic acid, phytic acid and derivatives thereof, ethylenediaminetetraacetic acid and derivatives thereof, diethylenetriaminepentaacetic acid and derivatives thereof, N-carboxymethyl-aspartic acid and derivatives thereof, N-carboxymethyl-glutamic acid and derivatives thereof, N,N-bis(carboxymethyl)-aspartic acid and derivatives thereof, N,N-bis(carboxymethyl)-glutamic acid and derivatives thereof, N,N-bis(succinate)-ethylenediamine and derivatives thereof, desfferioxamine, o-phenanthroline, transferrin, ferritin, lactoferrin, caffeic acid, maltol, purpurogalin, pyrogallol, sodium polyphosphate, sodium metaphosphate, sodium hexametaphosphate, and the like.

Examples of the oleaginous material include fats and oils such as animal and vegetable oils, waxes such as lanolin, hydrocarbons such as paraffin, higher alcohols such as cetanol, higher fatty acids such as stearic acid, sterols, phospholipids such as lecithin, synthetic esters such as myristic acid, metal soaps, silicone oil, perfluoropolymers, perfluoropolyethers, and the like.

Examples of the surfactant include anionic surfactants, cationic surfactants, nonionic surfactants, emulsifiers, solubilizers, and the like.

Examples of the solvent include lower alcohols such as ethanol, ethers, glycerins, liquid nonionic surfactants, liquid oleaginous materials, other organic solvents, water, and the like.

Examples of the polymeric substance include polyamino acids such as polyaspartic acid, ε-polylysine, γ-polyglutamic acid and derivatives thereof, naturally occurring polymer compounds such as collagen and elastin, semi-synthetic polymer compounds such as partially deacetylated chitin, synthetic polymer compounds such as carboxymethyl cellulose, and the like.

Examples of the powdery substance include inorganic pigments such as talc, functional pigments such as synthetic mica, particulate composite powders (hybrid fine powders), pearl-gloss pigments such as titanium dioxide-coated mica, photochromic pigments, polymer powders such as nylon powder, organic powders such as N-ε-lauroyl lysine, and the like.

Examples of the dye include Tar Dye Group I designated by law, Tar Dye Group II designated by law, Tar Dye Group III designated by law, hair dyes, natural dyes, mineral dyes, and the like.

Examples of the fragrance include fragrance from animals such as musk, fragrance from plants such as jasmine, synthetic fragrance such as α-amylcinnamaldehyde, composite fragrance, and the like.

Examples of the transdermal absorption promoting agent include urea, 2-pyrrolidone, 1-hexanol, 1-octanol, 1-decanol, 1-menthol, sodium laurylsulfate, isopropyl myristate, n-hexyl acetate, oleic acid, and the like.

Examples of the steroid hormone include 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, formocortal, halcinonide, halometasone, halopredone acetate, hydrocortamate, hydrocortisone, hydrocortisone phosphate, hydrocortisone-21-sodium succinate, hydrocortisone tebutate, mazipredone, medrysone, meprednisone, methylprednisolone, mometasone furoate, paramethasone, prednicarbate, prednisolone-21-diethylaminoacetate, prednisone sodium phosphate, prednisolone sodium succinate, prednisolone sodium-21-m-sulfobenzoate, prednisolone-21-stearoylglycolate, prednisolone tebutate, prednisolone-21-trimethylacetate, preidnisone, prednival, prednylidene, prednylidene-21-diethylaminoacetate, tixocortal, triamcinolone, triamcinolone acetonide, triamcinolone benetonide and triamcinolone hexacetonide, fluticasone, and the like.

The dosage form of the cosmetic or the skin preparation is not particularly limited, and includes dosage forms such as a solution, a paste, a gel, a solid, or a powder. Furthermore, the cosmetic or the skin preparation can be an oil, a lotion, a cream, an emulsion, a gel, a shampoo, a hair rinse, a hair conditioner, an enamel, a foundation, a lip stick, a face powder, a facial mask, an ointment, a tablet, an injection, a granule, a capsule, a perfume, a powder, an eau de Cologne, a dental paste, a soap, an aerosol and a cleansing foam, and additionally in an agent for preventing and improving skin aging, an agent for preventing and improving dermatitis, a bathing agent, a hair growth agent, a skin essence, an agent for preventing sunburn, an agent for preventing and improving light photosensitivity such as xeroderma pigmentosum and sunlight urticaria, an agent for preventing and improving photoallergy, an agent for preventing and improving photoinhibition of immunity, or an agent for preventing and improving rough skin caused by such as injuries, cracks and chaps, a disinfectant, an antibacterial agent, an insecticide, a pest control agent, a keratolytic agent, an agent for epidermal peeling, an agent for preventing and improving acne, an agent for preventing and improving various skin diseases such as keratosis, xeroderma, ichthyosis, and psoriasis, and the like.

Furthermore, other components which are commonly used in a cosmetic or a skin preparation for external use can be added to the cosmetic or the skin preparation containing the zinc salt of an acylamino acid or the zinc salt of an organic acid in an amount which does not inhibit the effect of the present invention. Examples of the other components commonly used in a cosmetic or a skin preparation for external use include a preservative, an agent for preventing browning, a buffer, an agent for acne, an agent for preventing dandruff or itching, an antiperspirant and deodorant agent, an agent for burn injury, an anti-mite and lice agent, an agent for softening keratin, an agent for xeroderma, an antiviral agent, a hormone, a vitamin, an amino acid, a peptide, a protein, an astringent agent, a freshening agent, a stimulating agent, an animal-derived component, a plant-derived component, an antibiotic, an antifungal agent, a hair growth agent, and the like.

### EXAMPLES

Hereinafter, the present invention will be described more specifically with reference to the following non-limiting Examples. In the Examples, the blending amounts are expressed as % by weight.

### Synthesis Example 1

### Synthesis of a Zinc Salt of lauroyl-L-glutamic acid, a Zinc Salt of Lauroyl Glycine, and a Zinc Salt of lauroyl-L-histidine

A zinc salt of lauroyl-L-glutamic acid was synthesized by the following method. Lauroyl-L-glutamic acid synthesized by a standard method (250 mg, 0.76 mmol) was dissolved in 10 ml of ethyl alcohol. An ethanol solution of 1% zinc acetate (Wako Pure Chemical Industries, Ltd.) was prepared separately. This 1% zinc acetate (18.4 ml) (0.84 mmol of zinc acetate, 1.1 equivalent weight of lauroyl-L-glutamic acid) was added to 10 ml of the previously prepared ethyl alcohol solution of lauroyl-L-glutamic acid, and the precipitate was separated by filtration. The precipitate was then washed with water, ethanol, and acetone, and then dried under reduced pressure. 200 mg of zinc salt of lauroyl-L-glutamic acid was obtained. The zinc salt of lauroyl glycine and the zinc salt of lauroyl-L-histidine were synthesized by reacting a lauroyl amino acid, synthesized in accordance with a standard method, with zinc acetate in the same manner as above, respectively. The results of elemental analysis of these compounds are shown in the Table 1.

TABLE 1

| | Composition (%) | | | | | | |
|---|---|---|---|---|---|---|---|
| | | | | | | | Ratio of |
| | | | | | | | lauroyl |
| | | | | | | | amino acid/ |
| Compound | | Carbon | Hydrogen | Nitrogen | Oxygen | Zinc | zinc |
| Zinc salt of | Calculated | 51.9 | 7.4 | 3.6 | 20.4 | 16.7 | 1.0 |
| lauroyl-L- | value | | | | | | |
| glutamic acid | Measured | 51.8 | 7.5 | 3.8 | 20.1 | 16.6 | |
| | value | | | | | | |
| Zinc salt of | Calculated | 58.2 | 9.0 | 4.8 | 16.6 | 11.3 | 1.9 |
| lauroyl | value | | | | | | |
| glycine | Measured | 58.1 | 9.0 | 5.1 | 16.3 | 12.0 | |
| | value | | | | | | |
| Zinc salt of | Calculated | 58.6 | 8.1 | 11.4 | 13.0 | 8.9 | 1.7 |
| lauroyl-L- | value | | | | | | |
| histidine | Measured | 55.0 | 8.1 | 11.2 | Not | 9.9 | |
| | value | | | | measured | | |

### Test Example 1

### The Effect of the Zinc Salt of Acylamino Acid on AP-1 Activation by Ultraviolet Light

To human dermal fibroblasts which had grown to confluence in a culture plate, a test compound was added in a concentration range which does not cause any damage to the fibroblasts. After 18 hours, the culture medium was replaced with a phenol red-free medium. By using Dermalei M-DMR-80 (manufactured by Toshiba Medical Supply Co., Ltd.), the fibroblasts were irradiated with ultraviolet light (UVA: 20 J/cm2). After 4 to 5 hours, the cells were collected, and a nuclear protein was extracted by a standard method. With regard to the obtained nuclear protein, activated AP-1 was detected by a gel shift assay. By measuring the radioactivity value of the AP-1 band using a bioimaging analyzer, BAS2000 (manufactured by Fuji Film Co., Ltd.), the amount of AP-1 was quantified.

The inhibition ratio of activation of AP-1 of the test compound was calculated by the following formula.

Inhibition ratio of activation of AP-1 (%)={1−(A1−A3)/(A2−A3)}×100

A1: radioactivity value of AP-1 band with addition of test compound

A2: radioactivity value of AP-1 band without addition of test compound

A3: radioactivity value of AP-1 band without addition of test compound and without ultraviolet irradiation

The results of the test compounds according to the present invention and the comparative compounds are shown in Table 2. The zinc salts of lauroyl amino acids exhibited a higher inhibition ratio compared with a zinc salt of an amino acid such as glycine or L-glutamic acid, which is known to prevent ultraviolet light by inducing metallothionein in the skin (for example, WO 9314748), or a zinc salt of a fatty acid such as lauric acid (for example, WO 0044341). These results show that the test compounds according to the present invention are able to significantly inhibit inflammation. Such an effect is not seen with the known zinc amino acid or zinc fatty acid.

TABLE 2

| | Concentration | |
|---|---|---|
| | for evaluation | |
| Test compound | (μM) | Inhibition ratio (%) |
| Zinc salt of N-lauroyl glycine | 50 | 59.2 |
| Zinc salt of N-lauroyl-L-glutamic | 50 | 89.6 |
| acid | | |
| Zinc salt of N-lauroyl-L- | 50 | 54.8 |
| histidine | | |

TABLE 3

| | Concentration | |
|---|---|---|
| | for evaluation | |
| Comparative compound | (μM) | Inhibition ratio (%) |
| Zinc salt of glycine | 50 | −72.8 |
| Zinc salt of L-glutamic acid | 50 | 2.4 |
| Zinc salt of lauric acid | 50 | 9.1 |

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A method of preventing, delaying, improving, or treating a skin inflammatory disease by applying to the skin a composition comprising a zinc salt of an acylamino acid derivative comprising: 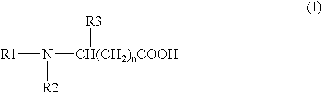
wherein, R1 represents an acyl group having 2 to 22 carbon atoms, R2 represents a hydrogen atom or a linear or branched alkyl group having 1 to 6 carbon atoms, R3 represents a side chain selected from the group consisting of valine, leucine, isoleucine, phenylalanine, methionine, tryptophan, asparagine, glutamine, serine, tyrosine, aspartic acid, glutamic acid, lysine, arginine, histidine, and hydrogen, and n represents an integer of 0 or 1.

2. A method of preventing, delaying, improving, or treating a skin inflammatory disease comprising applying to the skin a composition comprising a zinc salt of an acylamino acid comprising: 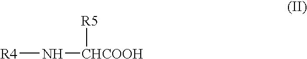
wherein, R4 represents an acyl group having 2 to 22 carbon atoms, R5 represents a side chain selected from the group consisting of valine, leucine, isoleucine, phenylalanine, methionine, tryptophan, asparagine, glutamine, serine, tyrosine, aspartic acid, glutamic acid, lysine, arginine, histidine, and hydrogen.

3. The method according to claim 2, wherein R4 is selected from the group consisting of octanoyl, decanoyl, lauroyl, myristoyl, palmitoyl, stearoyl, and cocoyl.

4. The method according to claim 2, wherein R5 is selected from the group consisting of glutamic acid, histidine, and hydrogen.

5. The method according to claim 2, wherein the composition is a preventive or therapeutic agent for a skin inflammatory disease.

6. The method according to claim 2, wherein the skin inflammatory disease is induced by ultraviolet light.

7. The method according to claim 2, wherein the composition is a cosmetic additive.

8. The method according to claim 2, wherein the composition is a skin preparation for external use.
